(19) United States
(12) Patent Application Publication
(10) Pub. No.: US 2024/0141315 A1
(43) Pub. Date: May 2, 2024

(54) COMPOSITIONS AND METHODS FOR RELEASE AND AMPLIFICATION OF NUCLEIC ACIDS

(71) Applicant: Nova Scientifix LLC, Henderson, NV (US)

(72) Inventor: Jimmy Wayne Fortenberry, Boaz, AL (US)

(21) Appl. No.: 18/384,825

(22) Filed: Oct. 27, 2023

Related U.S. Application Data
(60) Provisional application No. 63/420,169, filed on Oct. 28, 2022.

(51) Int. Cl. C12N 15/10; B01L 7/00

(52) U.S. Cl. CPC C12N 15/1003; B01L 7/52; B01L 2200/0647; B01L 2200/16; B01L 2300/0663

(57) ABSTRACT

Compositions, methods, kits, and systems are provided in which a biological sample containing cells and/or viruses that include a nucleic acid of interest are treated with an amplification-compatible releasing composition that provides release of the nucleic acid from cells and/or viruses in the sample and also permits amplification of the released nucleic acids without an intervening separation step. Methods incorporating such compositions are also described, and provide simple and rapid release of nucleic acids from cells and viruses along with subsequent amplification steps. Collection devices incorporating such compositions and kits for collection, release, and amplification of nucleic acids utilizing such compositions are provided, as are systems for characterizing nucleic acids.

This application claims the benefit of Provisional Patent Application No. 63/420,169 filed on Oct. 28, 2022. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is nucleic acid characterization.

### BACKGROUND

The following description includes information that may be useful in understanding the present invention. It is not an admission that any of the information provided herein is prior art or relevant to the presently claimed invention, or that any publication specifically or implicitly referenced is prior art.

Sequencing of DNA or RNA or identification of the presence of specific DNA or RNA sequences (e.g., in the detection of pathogens or identification of a genetic disease) typically involves release of nucleic acids from tissues, cells, and/or viruses present in a sample, followed by amplification of the released nucleic acids. There is considerable interest in developing and improving methods for performing these steps, however integration of them has proven challenging.

Typically, releasing nucleic acids from cells and/or viruses in a biological sample requires an isolation step. This isolation step separates nucleic acids from reagents utilized to release them into solution from encapsulating membranes and/or capsids. The reagents used to do so also tend to strongly inhibit enzymes utilized in subsequent amplification steps. Separation is typically accomplished in one of several ways. For example, nucleic acids are typically isolated by precipitating them from solution (e.g., by adding an alcohol) followed by centrifugation and collection of the resulting pellet. Other commonly used methods include using an insoluble phase to capture the released nucleic acids. Typical solid phases used for this purpose include filters and microparticles. All of these methods inherently involve loss of some of the nucleic acid present in the sample. They also involve a number of handling steps, which even in automated systems increase the risk of contamination. In addition, current methods for nucleic acid release can take considerable time (e.g., 15 minutes or more), which can hinder efforts that involve a large number of samples. This time requirement is further exacerbated by additional steps required for isolation of the released nucleic acids. Finally, such approaches can generate a significant stream of biohazardous waste (e.g., supernatants from precipitation reactions, used filters, used particles, pipette tips, centrifuge tubes, etc.).

Once released, characterization of nucleic acids almost invariably requires an amplification step. These involve the use of nucleic acid polymerases, free nucleotides, and primer nucleic acid sequences. Nucleic acid polymerases, however, are readily inactivated by reagents used to release nucleic acids from cells and/or viruses, which necessitates the implementation of isolation methods as described above. As a result, release and isolation of nucleic acids in sample are generally segregated from amplification steps, requiring a transfer of the isolated nucleic acid between release/isolation steps and amplification steps. This transfer step dramatically increases the chance of contamination of the amplification process, from different samples and from the environment. In addition, such segregation hampers the development of rapid and efficient automated systems for nucleic acid characterization.

Thus, there is still a need for rapid method for both extracting and amplifying nucleic acids from cells and/or viruses.

### SUMMARY OF THE INVENTION

The inventive subject matter provides compositions, methods, kits, and systems in which a biological sample containing cells and/or viruses that include a nucleic acid of interest are treated with an amplification-compatible extraction buffer that provides release of the nucleic acid from cells and/or viruses in the sample and also permits amplification of the released nucleic acids without an intervening separation (e.g., precipitation, filter capture and release, particle capture and release, etc.) step.

One embodiment of the inventive concept is a composition for release and amplification of a nucleic acid from a biological sample that includes a surfactant, an enzyme inhibitor; and a stabilizer. These are provided in amounts that release of the nucleic acid from the biological sample in less than about 10 minutes and that do not inhibit a polymerase chain reaction (for example, when present at greater than 10% by volume of the final amplification mixture). Suitable surfactants include NP-40, Triton X100, and/or SDS at a concentration of about 0.1% w/v to about 5% w/v. Suitable enzyme inhibitors include a magnesium chelating agent at a concentration of about 0.5 mM to about 10 mM. Suitable stabilizers including an albumin, a gelatin, a proanthocyanidin, and/or a salicylate at a concentration of about 0.1% w/v to about 10% w/v.

Another embodiment of the inventive concept is a composition for release and amplification of a nucleic acid from a cell and/or virus in a biological sample that includes a surfactant, an enzyme inhibitor; and a stabilizer. These are provided in amounts that release of the nucleic acid from the biological sample in less than about 10 minutes and that do not inhibit a polymerase chain reaction (for example, when present at greater than 10% by include acid salt, an alkyl ether, and/or a fatty alcohol polyoxyethylene ether at a concentration of about 0.1% w/v to about 20% w/v. Suitable enzyme inhibitors include a chaotrope and a magnesium chelating agent, where the chaotrope is from about 0.5 M to about 6 M and where the magnesium chelating agent is from about 1 mM to about 45 mM. Suitable stabilizers include a protein (e.g., an albumin and/or a gelatin), a proanthocyanidin, and/or a salicylate at concentration of from about 0.1% w/v to about 10% w/v.

Another embodiment of the inventive concept is a method of releasing a nucleic acid from a cell or virus in a sample and amplifying the nucleic acid, by adding at least a portion of the sample to a collection device that includes a composition as described above, incubating the portion of the sample for from 1 second to 10 minutes to generate an extracted nucleic acid solution in the composition, and adding a nucleic acid polymerase and a primer to the extracted nucleic acid solution without an intervening separation step. This generates an amplification mixture. In some embodiments the content of said composition in the amplification mixture is greater than about 10% v/v. In some embodiments incubating takes place for less than 1 minute. Some embodiments include a step of adding an amplification buffer to the extracted nucleic acid solution and/or the amplification mixture.

Embodiments of the inventive concept include a collection device that includes a reservoir, a closure, and a composition as described above held within the reservoir. In some embodiments the collection device is designed to permit thermal cycling of contents of the reservoir.

Embodiments of the inventive concept include a kit for collection and amplification of a nucleic acid, which include a collection device as described above and a sample collection device. Such a kit can also include a nucleic acid amplification primer.

Embodiments of the inventive concept include a system for amplifying a nucleic acid, which includes a collection and amplification reservoir containing a composition as described above and that is designed to receive a sample collection device. Such a system includes a reagent reservoir containing a nucleic acid polymerase and a liquid handling device designed and positioned to transfer liquid from the reagent reservoir to the collection and amplification reservoir. In some embodiments the system includes a second reagent reservoir that includes a nucleic acid amplification primer. In some embodiments the system can include a thermal cycler that is in thermal communication with the collection and amplification primer. In some embodiments the system can include an optical sensor, wherein the optical sensor that is in optical communication with the collection and amplification reservoir. In some embodiments the system does not include a separation device. For example, such a system can not include (i.e. specifically exclude) a centrifuge, filter, magnet, or other device utilized to separate solids (e.g. precipitates, colloids, particles, etc.) from a liquid phase.

Various objects, features, aspects and advantages of the inventive subject matter will become more apparent from the following detailed description of preferred embodiments, along with the accompanying drawing figures.

### BRIEF DESCRIPTION OF THE DRAWINGS

FIG. 1: FIG. 1 schematically depicts a prior art nucleic acid release and amplification method.

FIG. 2: FIG. 2 schematically depicts an exemplary method of the inventive concept.

### DETAILED DESCRIPTION

The inventive subject matter provides compositions, methods, kits, and systems in which a biological sample containing cells and/or viruses that include a nucleic acid of interest are treated with an amplification-compatible releasing composition that provides release of the nucleic acid from cells and/or viruses in the sample and also permits amplification of the released nucleic acids without an intervening separation (e.g., precipitation, filter capture and release, particle capture and release, etc.) step. Methods incorporating such compositions are also described, and provide simple and rapid release of nucleic acids from cells and viruses along with subsequent amplification steps, while minimizing opportunities for contamination and minimizing the production of biohazardous waste. Collection devices incorporating such compositions and kits for collection, release, and amplification of nucleic acids utilizing such compositions are provided, as are systems for characterizing nucleic acids.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The inventive subject matter provides compositions, methods, kits, and systems in which a biological sample containing cells and/or viruses that include a nucleic acid of interest are treated with an amplification-compatible extraction buffer that provides release of the nucleic acid from cells and/or viruses in the sample and also permits amplification of the released nucleic acids without an intervening separation (e.g., precipitation, filter capture and release, particle capture and release, etc.) step.

Compositions, methods, kits, and/or systems of the inventive concept can be applied to any suitable sample containing a cell or a virus that includes a nucleic acid. Suitable samples include, but are not limited to, sample swabs (e.g., swabs applied to mucosal surfaces, tissue or biopsy samples, tumor tissue, body fluids (e.g., saliva, urine, mucus, spinal fluid, synovial fluid, vitreous humor), and whole organisms. Such samples can be freshly obtained, stored at reduce temperatures (e.g., 2° C. to 8° C., −20° C., −80° C., or −200° C.), or preserved (e.g., by drying, lyophilization, and/or chemical treatment). Suitable cells include eukaryotic (e.g., vertebrate, invertebrate, fungal, bacterial, and/or mycoplasma cells) from culture. Suitable cells include DNA viruses, RNA viruses, encapsulated viruses, and non-encapsulated viruses. Suitable viruses include, but are not limited to, HIV, HPV, hepatitis A virus, hepatitis B virus, hepatitis C virus, herpes simplex, an influenza virus, and a coronavirus. It should be appreciated that such samples can include one or more pathogens, and that compositions of the inventive concept can inactivate pathogens present in the sample upon use. It should be appreciated that compositions described herein can kill or inactivate pathogens found in samples that are being characterized, and so are particularly useful for biomedical testing. Examples of pathogens suitable for treatment using compositions, methods, collection containers, kits, and systems of the inventive concept include (but are not limited) to those listed in Table 1.

TABLE 1

| Viral Pathogens | Bacterial/Fungal Pathogens |
|---|---|
| Adenovirus | Acinetobacter baumannii |
| Bocavirus | Alternaria spp. |
| Coronavirus | Aspergillus spp. |
| COVID 19 | Atopobium vaginae |
| EBV (mononucleosis) | Bacteroides fragilis |
| HHV-1 (Herpes Simplex) | Bordetella pertussis |
| HHV-2 (Herpes Simplex) | BVAB-2 |
| Human Metapneumovirus | Candida albicans |
| Influenza A & B | Candida dubliniensis |
| Parainfluenza Virus (type 1-4) | Candida glabrata |
| Respiratory Syncytial Virus A&B | Candida krusei |
| Rhinovirus | Candida lusitaniae |
| | Candida parapsilosis |
| | Candida tropicalis |
| | Chlamydia trachomatis |
| | Chlamydophila pneumoniae |
| | Citrobacter braakii/freundii |
| | Citrobacter koseri |
| | Cryptococcus spp. |
| | Curvularia spp. |
| | Enterobacter cloacae |
| | Enterococcus spp. |
| | Epidermophyton floccosum |
| | Escherichia coli |
| | Escherichia coli |
| | Fusarium spp. |
| | Gardnerella vaginalis |
| | Haemophilus ducreyi |
| | Haemophilus influenzae |
| | Klebsiella aerogenes |
| | Klebsiella oxytoca |
| | Klebsiella pneumoniae |
| | Lactobacillus crispatus |
| | Lactobacillus gasseri |
| | Lactobacillus iners |
| | Lactobacillus jensenii |
| | Legionella pneumophila |
| | Malassezia spp. |
| | Megasphaera Type 1 &2 |
| | Meyerozyma guilliermondii |
| | Microsporum canis |
| | Mobiluncus curtisii |
| | Mobiluncus mulieris |
| | Moraxella catarrhalis |
| | Morganella morganii |
| | Mycoplasma genitalium |
| | Mycoplasma hominis |
| | Mycoplasma pneumoniae |
| | Neisseria gonorrhoeae |
| | Prevotella bivis |
| | Proteus mirabilis |
| | Pseudomonas aeruginosa |
| | Sarocladium strictum |
| | Scytalidium dimidiatum |
| | Serratia marcescens |
| | Staphylococcus aureus |
| | Staphylococcus epidermidis |
| | Staphylococcus saprophyticus |
| | Streptococcus agalactiae (Grp B) |
| | Streptococcus pyogenes (Group A) |
| | Treponema pallidum |
| | Trichomonas vaginalis |
| | Trichomonas vaginalis |
| | Trichophyton anthropophilic spp. |
| | Trichophyton zoophilic spp. |
| | Trichosporon spp. |
| | Ureaplasma urealyticum |

Within this application the term “about” refers to a range of values that is within 10% of the cited value. For example, “about 10” refers to a range of values from 9 to 11.

Within this application, the terms “composition” and “releasing composition” are equivalent.

Compositions of the inventive concept can provide release of nucleic acid from a cell and/or virus in a sample on introducing the sample to the composition and allowing it to incubate for a suitable period of time. This suitable period of time can be less than 10 minutes (e.g., about 1 second, about 2 seconds, about 5 seconds, about 10 seconds, about 15 seconds, about 20 seconds, about 30 seconds, about 45 seconds, about 1 minute, about 1.5 minutes, about 2 minutes, about 3 minutes, about 4 minutes, about 5 minutes, about 6 minutes, about 7 minutes, about 8 minutes about 9 minutes, about 10 minutes, or any interval encompassed by these values). For example, in some embodiments the composition is effective to release nucleic acids from cells and/or viruses in the sample within about 2 minutes to about 3 minutes after the sample is brought into contact with compositions.

Such a composition for release and amplification of a nucleic acid from a biological sample can includes a surfactant, an enzyme inhibitor; and a stabilizer. These are provided in amounts that release the nucleic acid from the biological sample in in a time interval as described above and that do not significantly (e.g., more than 50%) inhibit the polymerase activity of enzymes utilized in a nucleic acid amplification method (e.g., a DNA polymerase and/or an RNA polymerase) when present at greater than about 10%, about 20%, about 30%, about 40%, about 50% about 60%, about 70% about 80%, about 90%, about 95%, about 98%, or about 98% by volume of the final amplification mixture). In some embodiments the composition does not significantly inhibit the nucleic acid polymerization activity of polymerases used in nucleic acid amplification reactions when such polymerases are added directly to the composition.

It should be appreciated that such composition advantageously permit amplification reactions to be performed in the same mixture used to release DNA or RNA from cells and/or viruses present in a sample, either through direct addition of amplification reaction reagents (e.g., a nucleic acid polymerase, free nucleotides, and one or more primers complementary to the nucleic acid of interest) or following slight dilution with a suitable buffer. This can eliminate the need to isolate released nucleic and to transfer the isolated nucleic acid to a separate amplification reaction mixture, which adds complexity, time, and opportunities for contamination to conventional methods.

In some compositions of the inventive concept suitable surfactants include nonionic surfactants such as polyethoxyethanol surfactants, octylphenol/polyethylene glycol-based surfactants, and polyoxyethylenesorbitan-based surfactants. Examples of these include NP40, Triton x-114, Triton X-100, Triton X-405, Tween 20, Tween 60, Tween 65, Tween 80, Span 40, and Span 80. Suitable surfactants can also be anionic surfactants, such as metal salts of C8 to C16 sulfonic or carboxylic acids (such as sodium octyl sulfate, sodium nonyl sulfate, sodium decyl sulfate, and sodium dodecyl sulfate. Suitable concentrations of suitable nonionic and anionic surfactant can range from about 0.1% w/v to about 5% w/v. For example, a suitable concentration of a suitable nonionic or anionic surfactant can be about 0.1% w/v, about 0.2% w/v, about 0.3% w/v, about 0.4% w/v, about 0.5% w/v, about 0.75% w/v, about 1% w/v, about 1.5% w/v, about 2% w/v, about 2.5% w/v, about 3% w/v about 3.5% w/v, about 4% w/v, about 4.5% w/v, about 5% w/v, or any intervening concentration.

Such compositions can include an enzyme inhibitor, for example a compound that inhibits DNAse and/or RNAse activity found in samples. Suitable enzyme inhibitors include a chelating agent selected to complex with divalent cations (e.g., calcium, magnesium, and/or zinc ions) that can act as cofactors. Suitable chelating agents include, but are not limited to, EDTA, EGTA, DTPA, tetrasodium glutamate diacetate, hydroxyiminodisuccinic acid, and trisodium ethylenediamine disuccinate, Suitable concentrations for such enzyme inhibitors can range from about 0.5 mM to about 10 mM. For example, a suitable concentration of a suitable enzyme inhibitor can be about 0.5 mM, about 1 mM, about 1.5 mM, about 2 mM, about 2.5 mM, about 3 mM, about 4 mM, about 5 mM, about 7.5 mM, about 10 mM, or any intervening concentration.

Such compositions can include a stabilizer, such as a protein. Suitable protein stabilizers include an albumin, a gelatin, a proanthocyanidin, and/or a salicylate. Suitable albumins include albumin obtained from mammalian serum (e.g., human serum albumin, bovine serum albumin, buffalo albumin sheep serum albumin, pig serum albumin, etc.), albumin obtained from reptile serum, and ovalbumin (e.g., chicken ovalbumin, turkey ovalbumin, quail ovalbumin, etc.). Such albumin stabilizing proteins can be present at a concentration of about at a concentration of about 0.1% w/v to about 10% w/v. For example a suitable concentration of an albumin stabilizer can be about 0.1% w/v, about 0.25% w/v, about 0.5% w/v, about 0.75% w/v, about 1% w/v, about 1.5% w/v, about 2% w/v, about 2.5% w/v, about 3% w/v, about 3.5% w/v, about 4% w/v, about 4.5% w/v, about 5% w/v, about 6% w/v, about 7% w/v, about 8% w/v, about 9%, w/v about 10% w/v or any intervening concentration, Suitable gelatins include teleost skin gelatin, deep-sea fish gelatin, bovine gelatin, horse gelatin, and pig gelatin. Suitable concentrations of gelatin stabilizer can range from about 0.1% w/v to about 3% w/v. For example, a suitable concentration of a gelatin stabilizer can be about 0.1% w/v, about 0.25% w/v, about 0.5% w/v, about 0.75% w/v, about 1% w/v, about 1.25% w/v, about 1.5% w/v, about 1.75% w/v, about 2% w/v, about 2.25% w/v, about 2.5% w/v, about 2.5% w/v, about 3% w/v, or any intervening value. Suitable proanthocyanidins include those obtained from berries (e.g., cranberries, blueberries), mangosteen, persimmon, cocoa, apple, coconut (e.g., coconut husk), and durian. Suitable concentrations of proanthocyanidin can be about 0.05% w/v to about 3% w/v. For example, a suitable concentration of proanthocyanidin can be about 0.05% w/v, about 0.1% w/v, about 0.2% w/v, about 0.5% w/v, about 0.75% w/v, about 1% w/v, about 1.25% w/v, about 1.5% w/v, about 1.75% w/v, about 2% w/v, about 2.5% w/v, or about 3% w/v Suitable salicylates include acetyl salicylic acid, salicin, choline salicylate, and salsalate. Suitable concentrations of salicylate can be about 0.05% w/v to about 8% w/v. For example, a suitable concentration of salicylate can be about 0.05% w/v, about 0.5% w/v, about 1% w/v, about 1.5% w/v, about 2% w/v, about 2.5% w/v, 3% w/v, about 4% w/v, about 5% w/v, about 6% w/v, about 7% w/v, or about 8% w/v.

Another composition for release and amplification of a nucleic acid from a cell and/or virus in a biological sample can provide long term (greater than 24 hours) stability of the released nucleic acid (e.g., DNA and/or RNA). Such long term stability can maintain at least 50% (i.e., 50% to 100%) of the released nucleic acid in a form and amount corresponding to that found immediately (e.g., 10 minutes or less) post-sample treatment for a period of 24 hours, 36 hours, 2 days, 3 days, 5 days, 1 week, 2 weeks, 3 weeks, 1 month, 2 months, 3 months, 4 months, 6 months 9 months, 1 year, 2 years, or more at ambient temperature. This advantageously provides significant temporal separation between release and amplification steps, and facilitates field collection of samples for mass screening.

Such a composition can include a surfactant, an enzyme inhibitor; and a stabilizer. These are provided in amounts that release of the nucleic acid from the biological sample in less than about 10 minutes and that do not inhibit a polymerase chain reaction. Suitable surfactants include, but are not limited to, Triton X 100, sodium octyl sulfate, sodium nonyl sulfate, sodium decyl sulfate, and sodium dodecyl sulfate, Tween 20, Tween 60, Tween 65, Tween 80, Span 40, and Span 80, sodium linear alkylbenzene sulfonate, sodium aliphatic alcohol polyoxyethylene ether sulfate, ammonium aliphatic alcohol polyoxyethylene ether sulfate, lauroyl glutamic acid, nonylphenol polyoxyethylene ether, Pingpingjia, glycerol monostearate, lignin sulfonate, heavy alkylbenzene sulfonate, alkyl sulfonate, alkyl polyether Fatty alcohol polyoxyethylene ether. Suitable surfactant concentrations can be from about 0.1% w/v to 20% w/v. For example a suitable surfactant concentration can be about 0.1% w/v, about 0.2% w/v, about 0.3% w/v, about 0.4% w/v, about 0.5% w/v, about 0.75% w/v, about 1% w/v, about 1.5% w/v, about 2% w/v, about 2.5% w/v, about 3% w/v about 3.5% w/v, about 4% w/v, about 4.5% w/v, about 5% w/v, about 7.5% w/v, about 10% w/v, about 12.5% w/v, about 15% w/v, about 17.5% w/v, about 20% w/v, or any intervening concentration

Suitable enzyme inhibitors for such compositions include a chaotrope and/or a divalent cation chelating agent. Suitable chaotropes include guanidinium, isothiocyanates (such as guanidinium isothiocyanate), urea, and thiourea. Such a chaotrope can be provided a concentration of from about 0.5 M to about 6 M. For example, a chaotrope can be provided at about 0.5 M, about 1 M, about 1.5 M, about 2 M, about 2.5 M, about 3 M, about 3.5 M, about 4 M, about 4.5 M, about 5 M, about 5.5 M, about 6 M, or any intervening value. Suitable divalent cation chelating agents can be selected to complex with divalent cations (e.g., calcium, magnesium, and/or zinc ions) that can act as cofactors. Suitable chelating agents include, but are not limited to, EDTA, EGTA, DTPA, tetrasodium glutamate diacetate, hydroxyiminodisuccinic acid, and trisodium ethylenediamine disuccinate, Suitable concentrations for such enzyme inhibitors can range from about 1 mM to about 50 mM. For example, a suitable concentration of a suitable divalent cation chelating agent can be about 1 mM, about 1.5 mM, about 2 mM, about 5 mM, about 7.5 mM, about 1 mM, about 5 mM, about 10 mM, about 15 mM, about 15 mM, about 20 mM, about 25 mM, about 30 mM, about 35 mM, about 40 mM, about 45 mM, about 50 mM, or any intervening value.

Such compositions can also include a stabilizer. Suitable stabilizers include a protein, (such as an albumin and/or a gelatin), a proanthocyandidin, and/or a salicylate. Suitable albumins include albumin obtained from mammalian serum (e.g., human serum albumin, bovine serum albumin, buffalo albumin sheep serum albumin, pig serum albumin, etc.), albumin obtained from reptile serum, and ovalbumin (e.g., chicken ovalbumin, turkey ovalbumin, quail ovalbumin, etc.). Such albumin stabilizing proteins can be present at a concentration of about at a concentration of about 0.1% w/v to about 10% w/v. For example a suitable concentration of an albumin stabilizer can be about 0.1% w/v, about 0.25% w/v, about 0.5% w/v, about 0.75% w/v, about 1% w/v, about 1.5% w/v, about 2% w/v, about 2.5% w/v, about 3% w/v, about 3.5% w/v, about 4% w/v, about 4.5% w/v, about 5% w/v, about 6% w/v, about 7% w/v, about 8% w/v, about 9%, w/v about 10% w/v or any intervening concentration, Suitable gelatins include teleost skin gelatin, deep-sea fish gelatin, bovine gelatin, horse gelatin, and pig gelatin. Suitable concentrations of gelatin stabilizer can range from about 0.1% w/v to about 3% w/v. For example, a suitable concentration of a gelatin stabilizer can be about 0.1% w/v, about 0.25% w/v, about 0.5% w/v, about 0.75% w/v, about 1% w/v, about 1.25% w/v, about 1.5% w/v, about 1.75% w/v, about 2% w/v, about 2.25% w/v, about 2.5% w/v, about 2.5% w/v, about 3% w/v, or any intervening value at concentration of from about 0.1% w/v to about 10% w/v. Suitable proanthocyanidins include those obtained from berries (e.g., cranberries, blueberries), mangosteen, persimmon, cocoa, apple, coconut (e.g., coconut husk), and durian. Suitable concentrations of proanthocyanidin can be about 0.05% w/v to about 3% w/v. For example, a suitable concentration of proanthocyanidin can be about 0.05% w/v, about 0.1% w/v, about 0.2% w/v, about 0.5% w/v, about 0.75% w/v, about 1% w/v, about 1.25% w/v, about 1.5% w/v, about 1.75% w/v, about 2% w/v, about 2.5% w/v, or about 3% w/v Suitable salicylates include acetyl salicylic acid, salicin, choline salicylate, and salsalate. Suitable concentrations of salicylate can be about 0.05% w/v to about 8% w/v. For example, a suitable concentration of salicylate can be about 0.05% w/v, about 0.5% w/v, about 1% w/v, about 1.5% w/v, about 2% w/v, about 2.5% w/v, 3% w/v, about 4% w/v, about 5% w/v, about 6% w/v, about 7% w/v, or about 8% w/v.

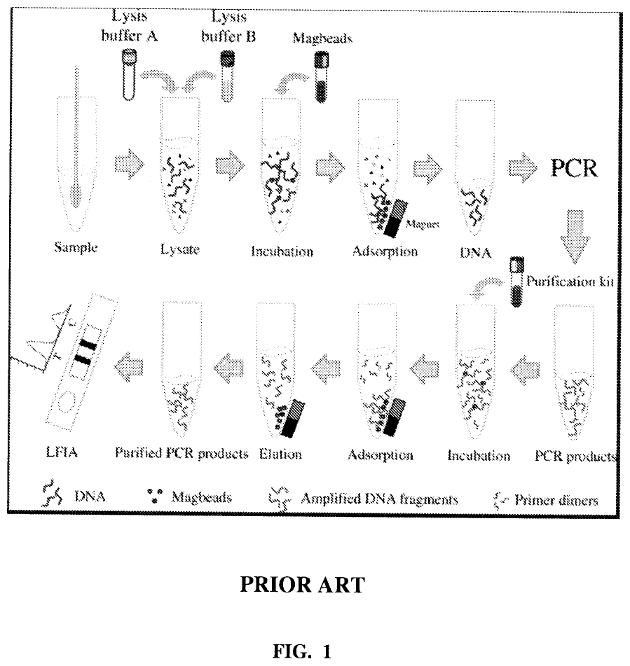

Another embodiment of the inventive concept is a method of releasing a nucleic acid from a cell or virus in a sample and amplifying the nucleic acid. FIG. 1 shows a typical prior art method for preparing a nucleic acid for an amplification step. As shown, a sample (in the form of a swab) is contacted with a two-part lysis solution that releases nucleic acids from cells of the sample. This typically requires from 30 minutes to several hours, or longer. A magnetic bead reagent (‘Magbeads’) is added. These magnetic beads are surface modified to bind to the released nucleic acids. A magnet is subsequently used to separate the nucleic acids from the lysis mixture, the liquid portion of which is removed and disposed of. The bound nucleic acid is subsequently released from the magnetic particles, The released nucleic acids are then transferred to a secondary tube for amplification (in this example, by polymerase chain reaction or PCR). The used magnetic particles are disposed of. In this example the products of PCR are captured on magnetic particles and subsequently released by magnetic separation and disposal of the used PCR reagents. These magnetic particles are similarly disposed of, along with the associated vessel. The purified PCR products can subsequently be analyzed (for example using a lateral flow device). It should be appreciated that each of these separation steps involve liquid handling, which provides an opportunity for contamination. In addition, separation, washing, and elution of the solid phase (in this instance magnetic beads) requires significant time. Finally, the method produces a significant amount of biohazardous waste.

In contrast, a method of releasing and amplifying nucleic acids from a sample that utilizes compositions of the inventive concept (e.g., as described above) begins by adding at least a portion of the sample to a collection device that includes a releasing composition as described above, incubating the portion of the sample for from 1 second to 10 minutes to generate an extracted nucleic acid solution in the releasing composition. For example, a sample can be incubated with the releasing composition for about 1 second, about 2 seconds, about 5 seconds, about 10 seconds, about 15 seconds, about 20 seconds, about 30 seconds, about 45 seconds, about 1 minute, about 1.5 minutes, about 2 minutes, about 3 minutes, about 4 minutes, about 5 minutes, about 6 minutes, about 7 minutes, about 8 minutes about 9 minutes, about 10 minutes, or any interval encompassed by these values. Amplification of the released nucleic acids can be accomplished by adding a nucleic acid polymerase, free nucleic acids, and a primer to the extracted nucleic acid solution without an intervening separation step. This generates an amplification mixture. In some embodiments a buffer can be added to adjust final concentrations of components in the amplification reaction mixture. In some embodiments the content of the releasing composition in the amplification mixture is from about 10% v/v to 99% v/v or greater. For example, the content of the releasing composition in the amplification mixture can be about 10% v/v, about 15% v/v, about 20% v/v, about 25% v/v, about 30% v/v, about 35% v/v, about 40% v/v, about 45% v/v, about 50% v/v, about 55% v/v, about 60% v/v, about 65% v/v, about 70% v/v, about 75% v/v, about 80% v/v, about 85% v/v, about 90% v/v, about 95% v/v, about 98% v/v, about 99% v/v, about 99.5% v/v, about 99% v/v, or any intervening value. In some embodiments incubating takes place for less than 1 minute. Some embodiments include a step of adding an amplification buffer to the extracted nucleic acid solution and/or the amplification mixture. The amplified nucleic acids can be subsequently characterized by any suitable method, including use of hybridization probes, mass spectrometry, hybridization to a microarray or bead array, etc.

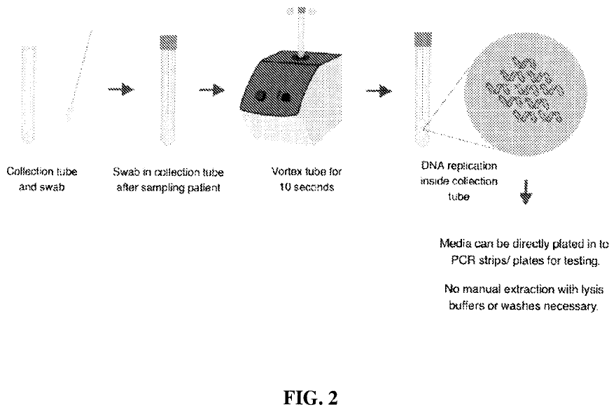

An example of a typical method for releasing and amplifying nucleic acids can be seen in FIG. 2. As shown, a sample (in this instance in the form of a swab) is placed in a collection device, which is can be closed with a closure to prevent contamination. The sample is incubated briefly (in this instance about 10 seconds) in the releasing composition, with optional mixing. Nucleic acid amplification reagents can be added to the tube or provided in combination with the releasing buffer, resulting in nucleic acid release and amplification (e.g., by PCR) taking place within the collection device, in this instance within about 10 seconds of sample addition. Following amplification, the contents of the collection vessel can be characterized, for example by using a PCR test strip, a test plate, or any suitable method. It should be appreciated such methods, which can be implemented using a releasing compositions of the inventive concept provides a greatly simplified and more rapid method for nucleic acid release, amplification, and characterization than is found in the prior art. In addition, such methods present far fewer opportunities for contamination and generate far less biohazardous waste than prior art methods.

Results of prior art methods and methods of the inventive concept are provided in Table 2. Table 2 shows a comparison of PCR results from samples treated with a Qiagen MagAttract™ HMW DNA Kit according to the manufacturer's instructions and a releasing composition of the inventive concept. Ct values obtained for a range of bacterial samples and viral samples are shown.

TABLE 2

| | Releasing | Qiagen | Percent |
|---|---|---|---|
| Sample | Composition | Extraction | Difference |
| Streptococcus pneumoniae | 27.029 | 27.394 | −0.013 |
| Staphylococcus aureus | 33.247 | 33.648 | −0.012 |
| Staphylococcus aureus | 22.015 | 25.374 | −0.132 |
| Staphylococcus aureus | 29.239 | 26.629 | 0.098 |
| Pseudomonas aeruginosa | 23.830 | 24.119 | −0.012 |
| Streptococcus pyogenes | 32.193 | 33.543 | −0.040 |
| Influenza A | 28.391 | 21.454 | 0.323 |
| Influenza A | 34.438 | 32.969 | 0.045 |
| Streptococcus pyogenes | 32.193 | 33.543 | −0.040 |
| Haemophilus influenzae | 26.167 | 26.849 | −0.025 |
| Haemophilus influenzae | 29.18 | 30.586 | −0.046 |

As shown, results from a method of the inventive concept are comparable to those of the Qiagen kit, despite being notably simpler to perform and allowing direct use in PCR.

Embodiments of the inventive concept include a collection device that includes a reservoir, a closure, and a releasing composition as described above held within the reservoir. In some embodiments the collection device is designed to permit thermal cycling of contents of the reservoir. For example, at least a portion of the collection device can be constructed of a thermally transmissive material positioned to be in contact with a compatible thermal cycler, or permit passage of microwave or other radiation that can be used for heating and thermal cycling. Alternatively, a collection vessel of the inventive concept. Similarly, such a collection device can be constructed of a material that permits transmission of wavelength utilized in monitoring an amplification reaction (e.g., excitation and emission wavelengths of fluorescent detection reagents, emission wavelengths of luminescent detection reagents, absorption wavelengths of chromogenic detection reagents, etc.). Such a collection device can be provided as an individual unit, or as part of an assembly of collection devices (e.g., a test plate). In some embodiments the collection device is configured for compatibility with a system for nucleic acid characterization. It should be appreciated that such a collection device can be sealed following insertion of the sample (e.g., with a closure), processed for release and amplification of nucleic acids while monitoring, and discarded as a unitary sealed device, thereby minimizing the production of biohazardous waste and reducing the chance of contamination.

Embodiments of the inventive concept include a kit for collection of a nucleic acid or collection an amplification of a nucleic acid. Such a kit includes a collection device as described above and a sample collection device. Such a kit can also include reagents for nucleic acid amplification, such as a nucleic acid polymerase, free nucleotides, and/or a primer. Such nucleic acid amplification reagents can be provided within the reservoir admixed with the releasing composition and/or in a separate container. In some embodiments such a kit can include a sample collection device, such as a swab, a bottle for collecting body fluids, and/or a tissue sampling device.

Embodiments of the inventive concept include a system for characterizing a nucleic acid. Such a system can include includes a collection and amplification reservoir containing a releasing composition as described above. This can be in the form of a collection device as described above. Alternatively, this reservoir can be in the form of a disposable reservoir that is provided with the releasing composition from a reagent reservoir of the system. Such a system can also include a reagent reservoir containing a nucleic acid polymerase, free nucleic acids, and/or amplification primers and a liquid handling device designed and positioned to transfer liquid from the reagent reservoir to the collection and amplification reservoir. Alternatively, such nucleic acid amplification reagents can be provided in the releasing composition. In some embodiments the system includes secondary reagent reservoirs that include nucleic acid amplification primer and/or probe sequences, which can be directed to different nucleic acids of interest. In some embodiments the system can include a device for agitating the contents of the collection and amplification reservoir. In some embodiments the system can include a thermal cycler that is in thermal communication with the collection and amplification reservoir. In some embodiments the system can include an optical sensor, wherein the optical sensor that is in optical communication with the collection and amplification reservoir. Such a system can include a controller and actuators (e.g., automated pipettors, decappers, etc.) that are in communication with a controller. Such a controller can include a processor capable of carrying out encoded instructions, and is in communication with a memory device. Such a memory device can include stored protocols that provide instructions for performing steps of nucleic acid release and amplification (for example, as shown in FIG. 2). In some embodiments the system does not include a separation device. For example, such a system can not include a centrifuge, filter, magnet, or other device normally utilized to separate solids (e.g. precipitates, colloids, particles, etc.) from a liquid phase.

One should appreciate that the compositions, methods, kits, and systems described herein advantageously simplify and streamline amplification of nucleic acids found in cells and/or viruses found in complex samples, by avoiding the necessity of separating nucleic acids of the samples from the reagents utilized for their release and doing so within a brief period of time (e.g., less than about 10 minutes, 8 minutes, 6 minutes, 5 minutes, 4 minutes, 3 minutes, 2 minutes, 1 minute, 45 seconds, 30 seconds, 20 seconds, 15 seconds, 10 seconds, 8 seconds, 6 seconds, 4 seconds, 2 seconds, 1 second, 0.8 seconds, 0.6 seconds, 0.5 seconds, 0.4 seconds, 0.3 seconds, 0.2 seconds, or 0.1 seconds).

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

what is claimed is:

1. A composition for release and amplification of a nucleic acid from a biological sample, comprising:
a surfactant in a first effective amount;
an enzyme inhibitor in a second effective amount; and
a stabilizer in a third effective amount,
wherein the first effective amount, the second effective amount, and the third effective amount provide release of the nucleic acid from the biological sample in less than about 10 minutes and do not inhibit a polymerase chain reaction.

2. The composition of claim 1, wherein the surfactant is selected from the group consisting of NP-40, Triton X100, and SDS, and wherein the first effective amount is from about 0.1% w/v to about 5% w/v.

3. The composition of claim 2, wherein the enzyme inhibitor is a magnesium chelating agent, and wherein the second effective concentration is from about 0.5 mM to about 10 mM.

4. The composition of claim 2, wherein the stabilizer is selected from the group consisting of an albumin, a gelatin, a proanthocyanidin, and a salicylate, wherein the third effective concentration is from about 0.1% w/v to about 10% w/v.

5. The composition of claim 1, wherein the surfactant is selected from the group comprising triton X 100, sodium dodecyl sulfate (SDS), tween 20, a linear alkyl benzene sulfonate sodium salt, fatty alcohol polyoxyethylene sulfate sodium salt, fatty alcohol polyoxyethylene sulfate ammonium salt, lauroyl glutamate, nonylphenol polyoxyethylene ether, Peregal, stearic acid monoglyceride, a lignin sulfonic acid salt, a heavy alkyl benzene sulfonic acid salt, an alkyl sulfonic acid salt, an alkyl ether, and a fatty alcohol polyoxyethylene ether, and wherein the first effective amount is from about 0.1% w/v to about 20% w/v.

6. The composition of claim 5, wherein the enzyme inhibitor is selected from the group consisting of is chaotrope and a magnesium chelating agent, wherein the second effective amount of the chaotrope is from about 0.5 M to about 6 M and wherein the second effective concentration of the magnesium chelating agent is from about 1 .M to about 45 mM.

7. The composition of claim 5, wherein the stabilizer is selected from the group consisting of an albumin, a gelatin, a proanthocyanidin, and a salicylate, wherein the third effective concentration is from about 0.1% w/v to about 10% w/v.

8. A method of releasing a nucleic acid from an organism in a sample and amplifying the nucleic acid, comprising
adding at least a portion of the sample to a collection device, wherein the collection device comprises a composition of claim 1; and
incubating the portion of the sample in the composition of claim 1 for from 1 second to 10 minutes to generate an extracted nucleic acid solution in the composition of claim 1; and
adding a nucleic acid polymerase and a primer to the extracted nucleic acid solution without an intervening separation step to form an amplification mixture,
wherein content of the composition of claim 1 in the amplification is greater than about 10% v/v.

9. The method of claim 8, wherein incubating is for less than 1 minute.

10. The method of claim 8, comprising adding an amplification buffer to the extracted nucleic acid solution or the amplification mixture.

11. A collection device comprising a reservoir, a closure, and the composition of claim 1, wherein the composition of claim 1 is enclosed by the reservoir.

12. The collection device of claim 11, wherein the collection device is configured to permit thermal cycling of contents of the reservoir.

13. A kit for collection and amplification of a nucleic acid, comprising:
a collection device of claim 11; and
a sample collection device.

14. The kit of claim 14, further comprising a nucleic acid amplification primer.

15. A system for amplifying a nucleic acid, comprising:
a collection and amplification reservoir comprising a composition of claim 1 and configured to receive a sample collection device;
a first reagent reservoir comprising a nucleic acid polymerase; and
a liquid handling device configured to transfer liquid from the reagent reservoir to the collection and amplification reservoir.

16. The system of claim 15, further comprising a second reagent reservoir comprising a nucleic acid amplification primer.

17. The system of claim 15, further comprising a thermal cycler in thermal communication with the collection and amplification primer.

18. The system of claim 15, further comprising an optical sensor, wherein the optical sensor is in optical communication with the collection and amplification reservoir.

19. The system of claim 15, wherein the system does not include a separation device.

20. The system of claim 19, wherein the separation device is selected from the group consisting of a centrifuge, a filter, and a magnet.
